(19) United States
(12) Patent Application Publication
(10) Pub. No.: US 2009/0246847 A1
(43) Pub. Date: Oct. 1, 2009

(54) METHOD FOR PRODUCING ETHANOL FROM STEAM EXPLODED SWEET POTATO BY FERMENTATION

(75) Inventors: HONGZHANG CHEN, BEIJING (CN); XIAOGUO FU, BEIJING (CN); WEIDONG WANG, CHANGSHA CITY (CN)

Correspondence Address: KNOBBE MARTENS OLSON & BEAR LLP, 2040 MAIN STREET, FOURTEENTH FLOOR, IRVINE, CA, 92614, US

(73) Assignees: INSTITUTE OF PROCESS ENGINEERING, CHINESE ACADEMY OF SCIENCES, BEIJING (CN); HU NAN QIANGSHENG MEDICINE CO. LTD., CHANGSHA CITY (CN)

(21) Appl. No.: 12/412,090

(22) Filed: Mar. 26, 2009

(30) Foreign Application Priority Data
Mar. 28, 2008 (CN) 200810102979.8
Mar. 28, 2008 (CN) 200810102980.0

(51) Int. Cl. C12P 7/06

(52) U.S. Cl. 435/161

(57) ABSTRACT

A method for producing ethanol from steam-exploded sweet potato by fermentation includes subjecting sweet potato to a steam explosion treatment, subjecting the sweet potato after the steam explosion to saccharification and fermentation, and collecting ethanol produced by the fermentation. The sweet potato can optionally be subjected to a pre-treatment utilizing a short-time, low-pressure steam explosion technology, which omits the long-time cooking process for the starch-based raw material and reduces the energy consumption for producing ethanol by fermentation. The method permits the steam explosion-treated sweet potato t be subjected directly to saccharification and solid-state fermentation, the water content in fermentation mash is reduced greatly so as to increase the concentration of ethanol in fermentation mash, reduce the energy consumption for distillation, reduce the subsequent treatment steps for the waste water and lower the producing cost, which are all beneficial to the comprehensive utilization of sweet potato.

### RELATED APPLICATIONS

This application claims the benefit of Chinese Patent Applications Nos. CN200810102980.0 and CN200810102979.8, both filed on Mar. 28, 2008, which are hereby expressly incorporated by reference in their entirety.

### FIELD OF THE DISCLOSURE

The present disclosure relates to a method for producing ethanol, and particularly, a method for producing ethanol from steam exploded sweet potato by fermentation.

### DESCRIPTION OF THE RELATED ART

With the rapid development of the human society, the energy source and resource crisis has become prominent increasingly, and therefore, the utilization of fuel ethanol obtained from the conversion of biomass raw materials has become a hotspot in the intensely competitive field of high tech research and industrialization in the world. Biological ethanol is produced by using different raw materials according to actual situations in different countries. For the development of biomass fuel ethanol on a basis of comprehensive consideration of the current practices and the conditions of the biological resources in China, as well as following a principle of “no reduction in foodstuffs for human beings and no reduction in croplands”, one feasible plan is to develop high yielding, aridity resistant, leanness resistant and foodstuff replaceable crops as raw materials for biomass fuels, by correspondingly adjusting measures to local conditions some areas which are unsuitable for growing high foodstuff-producing crops. For example, sweet potato, which grows widely in China and has a high productivity and a high starch content, is a potential raw material for producing fuel ethanol.

Sweet potato is originally produced in South America and was introduced to China more than 400 years ago. It is widely cultured in most provinces in China and the culturing area thereof is about 9,300 thousand hectares. The total output of sweet potato per year in China is about 0.45 billion tons which comprises 80% or more of the total output of sweet potato in the world. However, because the main producing areas of sweet potato in China are mostly needy, droughty mountainous areas with inconvenient traffic and behindhand processing, the sweet potato is ordinarily consumed in a manner of ⅓ for human consumption, ⅓ for feedstuff and ⅓ for corruption. Additionally, the processing of sweet potato is mostly carried out in small-scale processing plants for consuming the redundant sweet potato, and the products thereof are mostly the primary products such as rough starch, noodle, vermicelli and the like, resulting in a low utilizing value and a relatively price. The fresh sweet potato has a starch content of up to 15-20% and can become a starch-based raw material for producing ethanol instead of the foodstuff crops of corn, wheat and the like partially.

The starch type raw materials used for producing ethanol by fermentation are conventionally subjected to high temperature or low temperature cooking, gelatinization, liquidation and further saccharification or the like, and finally, after all the above steps, to ethanol fermentation, wherein the energy consumed by the cooking of the raw materials comprises 30-40% of the total energy consumption for producing ethanol.

The steam explosion treating technology (hereinafter briefly referred to as steam explosion technology) developed recently in the field of wood pulping is a technology which comprises steps of cooking the raw materials for a certain time using the media of water steam, air or the like under a certain pressure, then producing secondary steam upon abrupt ejection under reduced pressure and increasing the volume dramatically, and thereby, destroying the structure of the solid materials by the effect of mechanical force.

### SUMMARY

The present disclosure generally relates to a method for producing ethanol from steam exploded sweet potato by fermentation, comprising: subjecting sweet potato to a steam explosion treatment; subjecting the sweet potato after the steam explosion to saccharification and fermentation; and collecting ethanol produced by the fermentation. In one embodiment of the foregoing method, the steam explosion treatment may be performed in a steam explosion tank under a steam pressure ranging from about 0.5 MPa to about 0.8 MPa, and/or for about 2 minutes to about 4 minutes. In another embodiment of the method, the fermentation may be the solid-state fermentation. In some embodiments, the saccharification may be performed at about 55° C. to about 60° C., and/or for about 20 minutes to about 60 minutes. In some other embodiments, the fermentation may be performed for about 48 hours to about 60 hours, and/or under a condition of about 30° C. to about 35° C.

According to one aspect of the present disclosure, the fermentation may be carried out after the saccharification of the steam exploded sweet potato. In some embodiments, the method may further comprise adding glucoamylase. In some of such embodiments, an amount of the glucoamylase may range about 100 U to about 150 U glucoamylase/g dry steam exploded sweet potato. In some other embodiments, the method may further comprise adding (NH₄)₂SO₄, KH₂PO₄ and activated yeast, and the addition amount of (NH₄)₂SO₄ may be, for example, about 0.1 g to about 0.15 g (NH₄)₂SO₄/100 g sweet potato, the addition amount of KH₂PO₄ may be, for example, about 0.1 g to about 0.2 g KH₂PO₄/100 g sweet potato, and the addition amount of said yeast may be, for example, about 0.10 g to about 0.30 g yeast/100 g sweet potato.

According to another aspect of the present disclosure, the sweet potato after the steam explosion may be subjected to a simultaneous saccharification and solid-state fermentation. In some embodiments, the method may further comprise adding an glucoamylase, (NH₄)₂SO₄, KH₂PO₄ and activated yeast. In some of such embodiments, the addition amount of the (NH₄)₂SO₄ may be, for example, about 0.1 g to about 0.15 g (NH₄)₂SO₄/100 g sweet potato, the addition amount of the KH₂PO₄ may be, for example, about 0.10 g to 0.2 g KH₂PO₄/100 g sweet potato, and the addition amount of the yeast may be, for example, about 0.10 g to about 0.30 g yeast/100 g sweet potato. In some other embodiments, the method may further comprise adding glucoamylase and an amount of the glucoamylase may range, for example, from about 100 U to about 150 U glucoamylase/g dry steam exploded sweet potato. In some of foregoing embodiments, the fermentation may be performed for about 48 hours to about 60 hours under a condition of about 30° C. to about 35° C.

### DETAILED DESCRIPTION OF THE PREFERRED EMBODIMENT

An object of the present disclosure is to provide a method for producing ethanol from steam exploded sweet potato by fermentation, which applies a steam explosion technology to pre-treatment of sweet potato. In some embodiments, the sweet potato after being treated by utilizing low pressure steam explosion can be directly used for producing ethanol by fermentation, and therefore, the problem of high energy consumption for sweet potato pulverization and starch cooking and liquidation before producing ethanol by sweet potato starch fermentation is solved.

Additionally, in some other embodiments of the disclosure, the steam explosion treated sweet potato is directly subjected to saccharification and solid-state fermentation, as a result, the water content in fermentation mash is reduced greatly so as to increase the concentration of ethanol in fermentation mash, reduce the energy consumption for distillation, reduce the subsequent treatment steps for waste water and lower the producing cost, which is beneficial to the comprehensive utilization sweet potato.

The technical solution of some aspects of the disclosure is as follows:

A method for producing ethanol from steam exploded sweet potato fermentation provided in some embodiments used in connection with the disclosure, comprises: 1) subjecting sweet potato to a steam explosion treatment; 2) subjecting the sweet potato after the steam explosion to saccharification and fermentation; and 3) collecting ethanol produced by the fermentation.

In one embodiments, the steam explosion treatment may be performed in a steam explosion tank under a steam pressure of about 0.5 MPa to about 0.8 MPa for about 2 minutes to about 4 minutes.

In an aspect of the disclosure, to the sweet potato after the steam explosion is added glucoamylase in a ratio of about 100 U to about 150 U/g dry steam exploded sweet potato, and saccharification is carried out at about 55° C. to about 60° C. for about 20 minutes to 60 minutes; and then, (NH₄)₂SO₄, KH₂PO₄ and activated yeast are added, and fermentation is carried out under a condition of about 30° C. to about 35° C. for about 48 hours to 60 hours; and finally, ethanol produced by fermentation is collected by distillation.

The addition amount of (NH₄)₂SO₄ may be: about 0.1 g to about 0.15 g (NH₄)₂SO₄/100 g sweet potato.

The addition amount of KH₂PO₄ may be: about 0.1 g to 0.2 g KH₂PO₄/100 g sweet potato.

The addition amount of said yeast may be: about 0.10 g to 0.30 g activated dry yeast/100 g sweet potato.

In another aspect of the disclosure, glucoamylase, (NH₄)₂SO₄, KH₂PO₄ and activated yeast are added into the sweet potato after the steam explosion, and fermentation is carried out under a condition of about 30° C. to 35° C. for about 48 hours to 60 hours, and finally, ethanol produced by fermentation is collected by distillation.

The addition amount of said glucoamylase is: about 100 U to 150 U glucoamylase/g sweet potato.

The addition amount of said (NH₄)₂SO₄ is: about 0.1 g to 0.15 g (NH₄)₂SO₄/100 g sweet potato.

The addition amount of said KH₂PO₄ is: about 0.10 g to 0.15 g KH₂PO₄/100 g sweet potato.

The addition amount of said yeast is: about 0.1 g to 0.30 g activated dry yeast/100 g sweet potato.

In some embodiments of the present disclosure, sweet potato is used as a starch based raw material for producing ethanol by fermentation. Aiming at the shortage of high energy consumption in the pre-treatment of cooking, liquidation and the like during the process of producing fuel ethanol from starch-based raw material, sweet potato is subjected to a pre-treatment utilizing a short-time (e.g. for about 2 minutes to about 4 minutes) steam explosion technology, which omits the long-time cooking process (e.g. for about 30 minutes to about 120 minutes) of the starch-based raw material, reduces the energy consumption for producing ethanol by fermentation, and shortens the period of production. Furthermore, by pre-treating sweet potato utilizing the steam explosion technology, the cell walls of the plant tissues are broken due to the thermal, mechanical and chemical actions of the steam explosion, consequently, the gelatinization ratio is increased, which is favorable to the progress of the saccharification process.

Additionally, in other embodiments of the disclosure, the steam explosion treated sweet potato can be subjected to a simultaneous saccharification and solid-state fermentation, so that the saccharification section can be omitted during the fermentation process, and the energy consumption is reduced. Because the saccharification and the fermentation may be performed simultaneously, glucose will be utilized by yeast once being produced in the saccharification, and therefore, the glucose can be kept at a relatively low level, which is favorable for preventing bacteria contamination while relieving the suppressing effect on the product.

Furthermore, because the solid-state fermentation is utilized for producing ethanol, the water content in fermentation mash is reduced greatly, as a result, it can increase the concentration of ethanol in fermentation mash, and at the same time, reduce the energy consumption for distillation, reduce the subsequent treating process for waste water, and provide conditions for the secondary utilization of fermentation mash with a low producing cost, which is beneficial to the comprehensive utilization of sweet potato.

### EXAMPLES

The technical solution of some embodiments of the disclosure is further described below by means of examples. In the examples of such embodiments, the glucoamylase was obtained from Beijing Donghua Qiangsheng Biotechnology Co., Ltd., and the yeast was an activated dry yeast for fermentation obtained from Hubei Yichang Angel Yeast Co., Ltd.

### Example 1

500 g of sweet potato were weighted, cut into small pieces, put into a steam explosion tank, and treated under a water steam pressure of 0.5 MPa for 2.5 min; glucoamylase was added into the treated material (100 U glucoamylase/g dry steam exploded material), and after being saccharified for 40 min in a water bath maintained at 60° C., the mixture was cooled to 30° C. and sterilized with ultraviolet radiation for 20 min. In terms of the mass percentage relative to the steam exploded sweet potato, 0.1% (NH₄)₂SO₄, 0.1% KH₂PO₄ and 0.10% activated yeast were added, and the resultant was fermented under a condition of 30° C. for 60 h. Ethanol was collected by distillation. The alcohol obtained by distillation was measured by an alcohol meter and the alcohol concentration thereof was 8.89%.

### Example 2

500 g of sweet potato were weighted, cut into small pieces, put into a steam explosion tank, and treated under a water steam pressure of 0.6 MPa for 2.5 min; glucoamylase was added into the treated material (120 U glucoamylase/g dry steam exploded material), and after being saccharified for 60 min in a water bath maintained at 55 C., the mixture was cooled to 32° C. and sterilized with ultraviolet radiation for 15 min. In terms of the mass percentage relative to the steam exploded sweet potato, 0.15% (NH₄)₂SO₄, 0.1% KH₂PO₄ and 0.25% activated yeast were added, and the resultant was fermented under a condition of 32° C. for 54 h. Ethanol was collected by distillation. The alcohol obtained by distillation was measured by an alcohol meter and the alcohol concentration thereof was 9.27%.

### Example 3

500 g of sweet potato were weighted, cut into small pieces, put into a steam explosion tank, and treated under a water steam pressure of 0.7 MPa for 2.5 min; glucoamylase was added into the treated material (150 U glucoamylase/g dry steam exploded material), and after being saccharified for 40 min in a water bath maintained at 58° C., the mixture was cooled to 35° C. and sterilized with ultraviolet radiation for 18 min. In terms of the mass percentage relative to the steam exploded sweet potato, 0.15% (NH₄)₂SO₄, 0.15% KH₂PO₄ and 0.30% activated yeast were added, and the resultant was fermented under a condition of 35° C. for 48 h. Ethanol was collected by distillation. The alcohol obtained by distillation was measured by an alcohol meter and the alcohol concentration thereof was 12.47%.

### Example 4

500 g of sweet potato were weighted, cut into small pieces, put into a steam explosion tank, and treated under a water steam pressure of 0.8 MPa for 2.5 min; glucoamylase was added into the treated material (150 U glucoamylase/g dry steam exploded material), and after being saccharified for 50 min in a water bath maintained at 60° C., the mixture was cooled to about 34° C. and sterilized with ultraviolet radiation for 20 min. In terms of the mass percentage relative to the steam exploded sweet potato, 0.15% (NH₄)₂SO₄, 0.15% KH₂PO₄ and 0.25% activated yeast were added, and the resultant was fermented under a condition of 34° C. for 60 h. Ethanol was collected by distillation. The alcohol obtained by distillation was measured by an alcohol meter and the alcohol concentration thereof was 10.84%.

### Example 5

500 g of sweet potato were weighted, cut into small pieces, put into a steam explosion tank, and treated under a water steam pressure of 0.7 MPa for 3.0 min; glucoamylase was added into the treated material (120 U glucoamylase/g dry steam exploded material), and after being saccharified for 30 min in a water bath maintained at 57° C., the mixture was cooled to 35° C. and sterilized with ultraviolet radiation for 15 min. In terms of the mass percentage relative to the steam exploded sweet potato, 0.1% (NH₄)₂SO₄, 0.1% KH₂PO₄ and 0.20% activated yeast were added, and the resultant was fermented under a condition of 35° C. for 60 h. Ethanol was collected by distillation. The alcohol obtained by distillation was measured by an alcohol meter and the alcohol concentration thereof was 12.08%.

### Example 6

500 g of sweet potato were weighted, cut into small pieces, put into a steam explosion tank, and treated under a water steam pressure of 0.7 MPa for 4.0 min; glucoamylase was added into the treated material (150 U glucoamylase/g dry steam exploded material), and after being saccharified for 20 min in a water bath maintained at 58° C., the mixture was cooled to 35° C. and sterilized with ultraviolet radiation for 18 min. In terms of the mass percentage relative to the steam exploded sweet potato, 0.15% (NH₄)₂SO₄, 0.1% KH₂PO₄ and 0.15% activated yeast were added, and the resultant was fermented under a condition of 35° C. for 60 h. Ethanol was collected by distillation. The alcohol obtained by distillation was measured by an alcohol meter and the alcohol concentration thereof was 11.72%.

### Example 7

500 g of sweet potato were weighted, cut into small pieces, put into a steam explosion tank and treated tinder a water steam pressure of 0.5 MPa for 2.5 min; after being cooled to room temperature, the steam exploded sweet potato was sterilized with ultraviolet radiation for 20 min; and into the treated steam exploded material, glucoamylase (100 U glucoamylase/g dry steam exploded material), and (NH₄)₂SO₄, KH₂PO₄ and activated yeast were added, wherein the mass percentages of (NH₄)₂SO₄, KH₂PO₄ and activated yeast relative to the steam exploded sweet potato were 0.1%, 0.1% and 0.10%, respectively; and then the mixture was fermented for 60 h under a condition of 30° C. Ethanol was collected by distillation. The alcohol obtained by distillation was measured by an alcohol meter and the alcohol concentration thereof was 8.77%.

### Example 8

500 g of sweet potato were weighted, cut into small pieces, put into a steam explosion tank, and treated under a water steam pressure of 0.6 MPa for 2.5 min; after being cooled to room temperature, the steam exploded sweet potato was sterilized with ultraviolet radiation for 20 min; and into the treated steam exploded material, glucoamylase (120 U glucoamylase/g dry steam exploded material), and (NH₄)₂SO₄, KH₂PO₄ and activated yeast were added, wherein the mass percentages of (NH₄)₂SO₄, KH₂PO₄ and activated yeast relative to the steam exploded sweet potato were 0.15%, 0.1% and 0.25%, respectively; and then the mixture was fermented for 54 h under a condition of 32° C. Ethanol was collected by distillation. The alcohol obtained by distillation was measured by an alcohol meter and the alcohol concentration thereof was 9.31%.

### Example 9

500 g of sweet potato were weighted, cut into small pieces, put into a steam explosion tank, and treated under a water steam pressure of 0.7 MPa for 2.5 min; after being cooled to room temperature, the steam exploded sweet potato was sterilized with ultraviolet radiation for 20 min; and into the treated steam exploded material, glucoamylase (150 U glucoamylase/g dry steam exploded material), and (NH₄)₂SO₄, KH₂PO₄ and activated yeast were added, wherein the mass percentages of (NH₄)₂SO₄, KH₂PO₄ and activated yeast relative to the steam exploded sweet potato were 0.15%, 0.15% and 030%, respectively; and then the mixture was fermented for 48 h under a condition of 35° C. Ethanol was collected by distillation. The alcohol obtained by distillation was measured by an alcohol meter and the alcohol concentration thereof was 12.33%.

### Example 10

500 g of sweet potato were weighted, cut into small pieces, put into a steam explosion tank, and treated under a water steam pressure of 0.8 MPa for 2.5 min; after being cooled to room temperature, the steam exploded sweet potato was sterilized with ultraviolet radiation for 20 min; and into the treated steam exploded material, glucoamylase (150 U glucoamylase/g dry steam exploded material), and (NH₄)₂SO₄, KH₂PO₄ and activated yeast were added, wherein the mass percentages of (NH₄)₂SO₄, KH₂PO₄ and activated yeast relative to the steam exploded sweet potato were 0.15%, 0.15% and 0.25%, respectively; and then the mixture was fermented for 60 h under a condition of 34° C. Ethanol was collected by distillation. The alcohol obtained by distillation was measured by an alcohol meter and the alcohol concentration thereof was 10.96%.

### Example 11

500 g of sweet potato were weighted, cut into small pieces, put into a steam explosion tank, and treated under a water steam pressure of 0.7 MPa for 3.0 min; after being cooled to room temperature, the steam exploded sweet potato was sterilized with ultraviolet radiation for 20 min; and into the treated steam exploded material, glucoamylase (120 U glucoamylase/g dry steam exploded material), and (NH₄)₂SO₄, KH₂PO₄ and activated yeast were added, wherein the mass percentages of (NH₄)₂SO₄, KH₂PO₄ and activated yeast relative to the steam exploded sweet potato were 0.1%, 0.1% and 0.20%, respectively; and then the mixture was fermented for 60 h under a condition of 35° C. Ethanol was collected by distillation. The alcohol obtained distillation was measured by an alcohol meter and the alcohol concentration thereof was 11.82%.

### Example 12

500 g of sweet potato were weighted, cut into small pieces, put into a steam explosion tame, and treated under a water steam pressure of 0.7 MPa for 4.0 min; after being cooled to room temperature, the steam exploded sweet potato was sterilized with ultraviolet radiation for 20 min; and into the treated steam exploded material, glucoamylase (150 U glucoamylase/g dry steam exploded material), and (NH₄)₂SO₄, KH₂PO₄ and activated yeast were added, wherein the mass percentages of (NH₄)₂SO₄, KH₂PO₄ and activated yeast relative to the steam exploded sweet potato were 0.15%, 0.1% and 0.15%, respectively; and then the mixture was fermented for 60 h under a condition of 35° C. Ethanol was collected by distillation. The alcohol obtained by distillation was measured by an alcohol meter and the alcohol concentration thereof was 11.51%.

What is claimed is:

1. A method for producing ethanol from steam exploded sweet potato by fermentation, comprising:
subjecting sweet potato to a steam explosion treatment;
subjecting the sweet potato after the steam explosion to saccharification and fermentation; and
collecting ethanol produced by the fermentation.

2. The method according to claim 1, wherein the steam explosion treatment is performed in a steam explosion tank under a steam pressure ranging from about 0.5 MPa to about 0.8 MPa.

3. The method according to claim 1, wherein the steam explosion treatment is performed for about 2 minutes to about 4 minutes.

4. The method according to claim 1, wherein the fermentation is the solid-state fermentation.

5. The method according to claim 1, wherein, in said subjecting, the fermentation is carried out after the saccharification of the steam exploded sweet potato.

6. The method according to claim 1 further comprising adding glucoamylase.

7. The method according to claim 6, wherein an amount of the glucoamylase ranges about 100 U to about 150 U glucoamylase/g dry steam exploded sweet potato.

8. The method according to claim 1, wherein the saccharification is performed at about 55° C. to about 60° C.

9. The method according to claim 1, wherein the saccharification is performed for about 20 minutes to about 60 minutes.

10. The method according to claim 1 further comprising adding (NH₄)₂SO₄, KH₂PO₄ and activated yeast.

11. The method according to claim 10, wherein the addition amount of (NH₄)₂SO₄ is about 0.1 g to about 0.15 g (NH₄)₂SO₄/100 g sweet potato, the addition amount of KH₂PO₄ is about 0.1 g to about 0.2 g KH₂PO₄/100 g sweet potato, and the addition amount of said yeast is about 0.10 g to about 0.30 g yeast/100 g sweet potato.

12. The method according to claim 1, wherein the fermentation is performed for about 48 hours to about 60 hours

13. The method according to claim 1, wherein the fermentation is performed under a condition of about 30° C. to about 35° C

14. The method according to claim 1, wherein, in said subjecting, the sweet potato after the steam explosion is subjected to a simultaneous saccharification and solid-state fermentation.

15. The method according to claim 14, wherein, in said subjecting, an glucoamylase, (NH₄)₂SO₄ KH₂PO₄ and activated yeast are added.

16. The method according to claim 15, wherein the addition amount of the (NH₄)₂SO₄ is about 0.1 g to about 0.15 g (NH₄)₂SO₄/100 g sweet potato, the addition amount of the KH₂PO₄ is about 0.10 g to 0.2 g KH₂PO₄/100 g sweet potato, and the addition amount of the yeast is about 0.10 g to about 0.30 g yeast/100 g sweet potato.

17. The method according to claim 14 further comprising adding glucoamylase

18. The method according to claim 17, wherein an amount of the glucoamylase ranges from about 100 U to about 150 U glucoamylase/g dry steam exploded sweet potato.

19. The method according to claim 14, wherein the fermentation is performed for about 48 hours to about 60 hours under a condition of about 30° C. to about 35° C.
